SEQUENCE LISTING (13 sequences)

SEQ ID NO: 1 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 2 - DNA, 59 bp, Artificial
  shMDR1(sense)
gatcccggcctaatgccgaacacatcgaaatgtgttcggcattaggccttttttccaac

SEQ ID NO: 3 - DNA, 59 bp, Artificial
  shMDR1(antisense)
tcgagttggaaaaaaggcctaatgccgaacacatttcgatgtgttcggcattaggccgg

SEQ ID NO: 4 - protein, 376 aa, Human herpesvirus 1
MASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGAAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - DNA, 1304 bp, Human herpesvirus 1
ataccgagcgaccctgcagcgacccgcttaacagcgtcaacagcgtgccgcagatcttggtggcgtgaaactcccgcacctcttcggccagcgccttgtagaagcgcgtatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggacgtcttggccaaacgcctccgtcccatgcatgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaa

SEQ ID NO: 6 - RNA, 19 nt, Artificial
  target sequence for shRNA
ggccuaaugccgaacacau

SEQ ID NO: 7 - DNA, 26 bp, Artificial
  HSV-tk sense
aaaagctagccttggtggcgtgaaac

SEQ ID NO: 8 - DNA, 26 bp, Artificial
  HSV-tk antisense
aaaaggatccgagttagcctccccca

SEQ ID NO: 9 - DNA, 32 bp, Artificial
  Nhe-Sal sense
aaaagctaccgtcgactagataactgaacttg

SEQ ID NO: 10 - DNA, 22 bp, Artificial
  Nhe-Sal antisense
tcttgatcagatccgaaaatgg

SEQ ID NO: 11 - DNA, 31 bp, Artificial
  TK-GFP antisense
aaaagtcgacttacttgtacagctcgtccat

SEQ ID NO: 12 - DNA, 19 bp, Artificial
  MDR1 sense
ggagtgtccgtggatcaca

SEQ ID NO: 13 - DNA, 23 bp, Artificial
  MDR1 antisense
aatacatcattgcctgggtgaag

(19) United States
(12) Patent Application Publication
(10) Pub. No.: US 2009/0214435 A1
(43) Pub. Date: Aug. 27, 2009

(54) Recombinant vector expressing MDR1 shRNA and thymidine kinase and use thereof

(75) Inventors: In San Kim, Daegu (KR); Seung Yoon Park, Gyeongbuk (KR); Jae Tae Lee, Daegu (KR); Byeong Cheol Ahn, Deagu (KR)

Correspondence Address: IPHORGAN, LTD., 1130 LAKE COOK ROAD, SUITE 240, BUFFALO GROVE, IL, 60089, US

(73) Assignee: Kyungpook National University Industry-Academic Cooperation Foundation

(21) Appl. No.: 12/077,215

(22) Filed: Mar. 18, 2008

(30) Foreign Application Priority Data
Apr. 6, 2007 (KR) 10-2007-0034241

(51) Int. Cl. A61K 31/7088; C12N 15/00; C12N 5/08; C12Q 1/02

(52) U.S. Cl. 424/9.6; 435/320.1; 435/366; 435/29; 514/44 R; 514/34; 514/510; 514/283

(57) ABSTRACT

A recombinant vector capable of expressing MDR1 shRNA and thymidine kinase, and a use thereof. More specifically, provided is a recombinant vector capable of efficiently expressing MDR1 shRNA and thymidine kinase in a host cell, a transfectant cell comprising the same recombinant vector, a composition for treating neoplastic diseases, comprising the same recombinant vector, and a method for imaging of neoplastic lesions using the same recombinant vector. The recombinant vector of the present invention is capable of achieving efficient intracellular expression of MDR1 shRNA and a thymidine kinase-GFP fusion protein within the host cell and is therefore highly effective for combined therapy of anticancer drugs. Further, the recombinant vector of the present invention enables imaging of neoplastic lesions. Therefore, the recombinant vector of the present invention can be used in combination with other anticancer drugs for treatment of neoplastic diseases.

### BACKGROUND OF THE INVENTION

1. Field of the Invention

The present invention relates to a recombinant vector capable of expressing MDR1 shRNA and thymidine kinase, and a use thereof. More specifically, the present invention relates to a recombinant vector capable of efficiently expressing MDR1 shRNA and thymidine kinase in a host cell, a transfectant cell comprising the same recombinant vector, a composition for treating neoplastic diseases, comprising the same recombinant vector, and a method for imaging of neoplastic lesions using the same recombinant vector.

2. Description of the Related Art

Cancer is a complex disease characterized by uncontrolled division and abnormal growth of malignant cells. Most of cancers result from various pathogenic factors including the genetic and epigenetic alterations of oncogenes and tumor suppressor genes. Cancer cells initially proliferate, then invade and destroy adjacent tissues, and finally spread into the circulatory system and metastasize to distant sites where they can continue their destructive processes, thereby resulting in death of individuals.

Various therapeutic approaches, such as surgical operation, radiotherapy, and chemotherapy, are employed to combat against such cancer diseases. Further, numerous ingredients known to have anticancer effects and derived from various sources are used for treatment of cancer. However, most of chemical anticancer drugs exhibit cytotoxicity on normal cells. To this end, there is a continued need for development of a novel anticancer therapy.

Further, since cancer is characterized by the propensity of tumor cells to spread from the primary lesion site to other normal tissues, the development and spread (i.e., metastasis) of the cancer disease leads to high mortality of cancer patients, despite remarkable advancement in the anticancer therapy including surgical operation, radiotherapy, and chemotherapy. Therefore, there is also a need for development of a tumor localization method, such as an imaging method for efficient detection of metastatic lesions of these tumor cells.

Thymidine kinase is an enzyme which catalyzes the ATP-dependent phosphorylation of deoxythymidine into deoxythymidine monophosphate and is implicated in various biosynthetic processes including DNA synthesis. It is present in two forms in mammalian cells, TKI and TKII. Certain viruses also have genetic information for expression of viral thymidine kinases. Further, thymidine kinase may also be used as a selective marker of transfectants. For this purpose, thymidine kinase is used in conjunction with ganciclovir which acts as a competitive inhibitor of guanosine through the action of thymidine kinase and other enzymes.

Meanwhile, P-glycoprotein is a membrane protein belonging to the ATP-binding cassette transporter (Ambudkar, S. V. et al., Oncogene, 22:7468-7485, 2003). P-glycoprotein is implicated in drug resistance against unrelated drugs which have a similar chemical structure but exhibit different specificity for target materials, such as paclitaxel, doxorubicin, and vincristine. P-glycoprotein-associated drug resistance is thought to be one of the obstacles in cancer chemotherapy. In order to overcome problems associated with the development of drug resistance, a variety of therapeutic substances, such as antibodies, antisense oligonucleotides, ribozymes, transcription factors, and the like, have been used for anticancer therapy (Mechetner, E. B. et al., Proc Natl Acad Sci USA, 89:5824-5828, 1992; Holm, P. S. et al., Br J Cancer, 70:239-243, 1994; Cucco, C. et al., Cancer Res, 56:4332-4337, 1996; and Marthinet, E. et al., Gene Therapy 7:1224-1233, 2000). Unfortunately, therapeutic effects of these substances on cancer are insignificant.

Recently, inhibition of gene expression using double-stranded RNA has emerged as an effective inhibition technique of gene expression in multicellular organs such as human cells. The principle of RNA interference (RNAi) technique is based on the nucleotide sequence-specific interaction between mRNA and small interfering RNA (siRNA). Long double-stranded RNA is decomposed into siRNA via the action of double-stranded RNA-specific RNase III Dicer, and the double-stranded siRNA or intracellularly expressed short hairpin RNA (shRNA) is integrated into an RNA-induced silencing complex (RISC). Thereafter, double-stranded siRNA is separated into single-stranded RNA molecules, and then the antisense strand binds to a target mRNA in a nucleotide sequence-specific manner, which consequently results in cleavage and destruction of the target mRNA to thereby inhibit RNA expression (Nykanen, A. et al., Cell, 107: 1090-1098, 2001).

### SUMMARY OF THE INVENTION

As a result of a variety of extensive and intensive studies and experiments to solve the problems as described above and develop an effective method for treatment of tumor, the inventors of the present invention discovered that the combined therapy of ganciclovir and an anticancer drug can be achieved to maximize anti-tumor effects by cloning of a thymidine kinase gene and MDR1 siRNA into one vector system and consequent intracellular incorporation of such a vector into a host cell, and it is also possible to obtain a nuclear medical image of tumor lesions by means of thymidine kinase-green fluorescent protein (TK-GFP) fusion gene inserted into the vector. The present invention has been completed based on these findings.

Therefore, the present invention has been made in view of the above problems, and it is an object of the present invention to provide a recombinant vector capable of expressing MDR1 shRNA and thymidine kinase, and a use thereof.

In accordance with an aspect of the present invention, the above and other objects can be accomplished by the provision of a recombinant vector capable of expressing MDR1 shRNA and thymidine kinase.

In accordance with another aspect of the present invention, there is provided a cell line transfected with the aforesaid recombinant vector.

In accordance with a further aspect of the present invention, there is provided a composition for preventing and/or treating neoplastic diseases, comprising the aforesaid recombinant vector.

In accordance with yet another aspect of the present invention, there is provided a method for imaging a neoplastic lesion, using the aforesaid recombinant vector.

### BRIEF DESCRIPTION OF THE DRAWING FIGURES

FIG. 1 illustrates an expression construct (A) and a cleavage map (B) of shMDR-TK-GFP recombinant vector in accordance with the present invention (P_{U6}: U6 promoter, P_{CMV}: CMV promoter, TK: thymidine kinase, GFP: green fluorescent protein, P_{SV40}: SV40 promoter, and Neo^{r}: neomycin resistance gene);

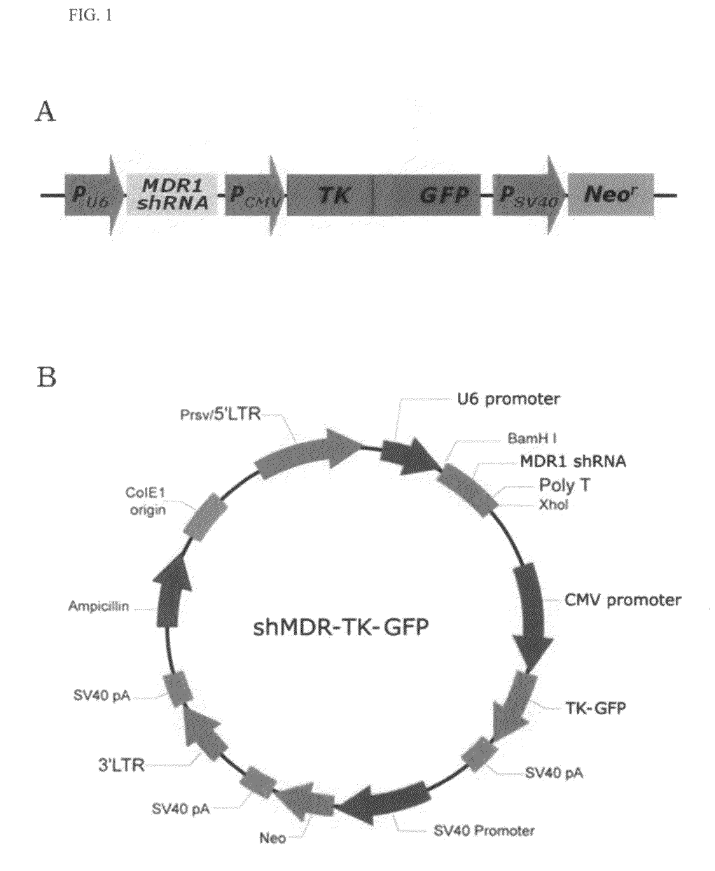

FIG. 2 illustrates confirmation of decreased MDR1 expression in response to expression of MDR1 shRNA, in a transfectant cell of the present invention. A: RNA level, and B: Protein level;

FIG. 3 illustrates confirmation of expression of a thymidine kinase-GFP fusion protein in a transfectant cell of the present invention. A: Protein level, and B: Fluorescence micrograph;

FIG. 4 illustrates test results for anticancer drug (paclitaxel) accumulation capacity (A) and anticancer drug (doxorubicin (B), paclitaxel (C)) sensitivity of a transfectant cell in accordance with the present invention;

FIG. 5 illustrates test results for ganciclovir accumulation capacity (A) and ganciclovir sensitivity (B) of a transfectant cell in accordance with the present invention; and

FIG. 6 illustrates combined therapeutic effects of ganciclovir and anticancer drug on a transfectant cell of the present invention (Dox: doxorubicin).

### DETAILED DESCRIPTION OF THE PREFERRED EMBODIMENTS

Hereinafter, the present invention will be described in more detail.

A recombinant vector of the present invention is characterized by expression of MDR1 shRNA and thymidine kinase.

P-glycoprotein is an expression product of the multidrug resistance 1 (hereinafter, referred to as MDR1) gene. P-glycoprotein is a transmembrane protein that acts as an energy-dependent efflux pump to remove natural drugs from cells. P-glycoprotein is normally present in the apical membrane of some epithelial cells in renal proximal tubules, hepatic bile ducts, and colon villi, but its function in these cells is not clear (Crodon-Cardo, C. et al., Cytochem, 38:1277-1287). Preferably, MDR1 may have an amino acid sequence as set forth in SEQ ID NO: 1, and may be, for example, one having Genbank Accession No. P08183, NP_{—}000918, CAA41558, AAB70218, AAB69423, AAR99172, AAA59575, AAA59576, AAR91622, or AAR91621.

shRNA is a single-stranded molecule of 50 to 70 nucleotides in length and forms a stem-loop structure in vivo. A 5- to 10-nucleotide loop connects the two complementary 19- to 29-nucleotide-long RNA fragments that create the double-stranded stem by base pairing. Transcription and synthesis of shRNA in vivo is directed by Pol III promoter, and then the resulting shRNA is cleaved by Dicer, an RNase III enzyme, to generate mature siRNA. The mature siRNA enters the RISC complex. Preferably, shRNA for inhibition of MDR1 expression in accordance with the present invention may contain both sense and antisense nucleotide sequences for a nucleotide sequence of SEQ ID NO: 6, and an insert fragment which allows expression of shRNA for the aforesaid target sequence (SEQ ID NO: 6) in an expression vector of the present invention have a sense nucleotide sequence as set forth in SEQ ID NO: 2 and an antisense nucleotide sequence as set forth in SEQ ID NO: 3. It was confirmed that such shRNA of the present invention is capable of efficiently inhibiting expression of MDR1 (see Example 2).

Further, thymidine kinase may preferably have an ammo acid sequence as set forth in SEQ ID NO: 4 or a nucleotide sequence as set forth in SEQ ID NO: 5, and may be, for example, one having Genbank Accession No. AAP13943, PO3176, AAA45811, PO4407, Q9QNF7, KIBET3, P17402, PO6478, PO6479, AAB30917, P08333, BAB84107, AAP13885, AAL73990, AAG40842, BAB11942, NP_{—}044624, NP_{—}044492, or CAB06747.

For expression of MDR1 shRNA and thymidine kinase, a promoter is operatively linked to a gene sequence encoding a protein of interest. As used herein, the term “promoter” refers to a DNA sequence that regulates expression of the target gene sequence being operatively linked to the promoter sequence in a certain host cell. The term “operatively linked” means that one nucleic acid fragment is linked to another nucleic acid fragment so that the function or expression thereof is affected by the other nucleic acid fragment. The expression cassette of the present invention may further comprise various expression regulatory sequences such as an optional operator sequence for controlling transcription, a sequence encoding a suitable mRNA ribosome-binding site, and sequences controlling the termination of transcription and translation. The promoter used in the present invention may be a constitutive promoter that constitutively induces the expression of a target gene, or an inducible promoter that induces the expression of a target gene at a given position and time point. Specific examples of the promoter may include U6 promoter, CMV (cytomegalovirus) promoter, SV40 promoter, CAG promoter (Hitoshi Niwa et al., Gene, 108:193-199, 1991; and Monahan et al., Gene Therapy, 7:24-30, 2000), CaMV 35S promoter (Odell et al., Nature 313:810-812, 1985), Rsyn7 promoter (U.S. patent application Ser. No. 08/991,601), rice actin promoter (McElroy et al., Plant Cell 2:163-171, 1990), ubiquitin promoter (Christensen et al., Plant Mol. Biol. 12:619-632, 1989), ALS promoter (U.S. patent application Ser. No. 08/409,297) and the like. Also usable promoters are disclosed in U.S. Pat. Nos. 5,608,149, 5,608,144, 5,604,121, 5,569,597, 5,466,785, 5,399,680, 5,268,463, 5,608,142, etc.

The promoters for expression of MDR1 shRNA and thymidine kinase may be identical or different from each other. The promoter may be appropriately controlled depending upon kinds of expression host cells and expression levels of target genes. For convenience, as used herein, the promoter for expression of MDR1 shRNA is designated as a first promoter, and the promoter for expression of thymidine kinase is designated as a second promoter.

Examples of the vector that can be used in the present invention may include, but are not limited to, plasmid, cosmid, bacteriophage and viral vectors. An optimal expression vector may comprise expression regulatory elements such as a promoter, an operator, an initiation codon, a stop codon, a polyadenylation signal and an enhancer, and may be prepared in various forms, depending upon desired applications and uses. Preferably, the expression vector of the present invention may be shMDR-TK-GFP.

The shMDR-TK-GFP vector was constructed according to the following procedure.

In order to construct a fusion gene of a Herpes simplex virus-thymidine kinase (HSV-tk) gene and a GFP (green fluorescent protein) gene (hereinafter, referred to as “TK-GFP gene”), PCR amplification was carried out using HSV-tk cDNA (by courtesy of Dr. Jae Yong Park, Department of Internal Medicine, Division of Pulmonary, Medicine College of Kyungpook National University, Korea) as a template, and primers having sequences as set forth in SEQ ID NOS: 7 and 8. PCR was carried out as follows: initial denaturation of template cDNA at 94° C. for 2 min, followed by 30 cycles of denaturation at 94° C. for 30 seconds, annealing at 60° C. for 30 seconds and extension at 72° C. for 1 min. The amplified PCR product was cleaved with Nhe I and BamH I restriction endonucleases and ligated into the same restriction sites (Nhe I and BamH I recognition sites) of the pEGFP-C1 vector (Clontech, USA), using T4 ligase (Invitrogen, USA), thereby constructing a recombinant vector, designated pTK-GFP.

First, in order to construct a vector where MDR1 shRNA is expressed, a nucleotide sequence (5′-GGCCUAAUGCCGAACACAU-3′) of SEQ ID NO: 6 was used as a target sequence. In order to construct an insert fragment which allows expression of shRNA for the aforesaid target sequence in an expression vector of the present invention, a pair of primers (SEQ ID NOS: 2 and 3) were designed. The primer sequences of SEQ ID NOS: 2 and 3 were designed to contain both sense and antisense nucleotide sequences for a nucleotide sequence of SEQ ID NO: 6, and insert a nucleotide sequence consisting of 4 bases (CGAA) therebetween to form a loop.

The thus-designed primer set was synthesized by Bionics (Seoul, Korea). Then, each 200 pmol of the primer fragments as set forth in SEQ ID NO: 2 and 3 was dissolved in STE buffer (10 mM Tris pH 8.0, 50 mM NaCl, 1 mM EDTA). The resulting solution was heated at 95° C., followed by slow cooling to allow binding of two primers, thereby constructing an insert fragment. The thus-formed insert fragment was inserted into BamH I and Xho I restriction sites of pRNAT/U6 vector (GenScript, USA), using T4 ligase (Invitrogen, USA), thereby constructing a recombinant vector, designated pRNAT/shMDR.

In order to make a Sal I restriction site for insertion of a TK-GFP gene simultaneously with removal of a GFP (Green fluorescent protein) gene present in the pRNAT/shMDR vector, PCR amplification was carried out using a pair of primers (SEQ ID NOS: 9 and 10) and the pRNAT/shMDR vector as a template. PCR was carried out as follows: initial denaturation of template DNA at 94° C. for 2 min, followed by 30 cycles of denaturation at 94° C. for 30 seconds, annealing at 60° C. for 30 seconds and extension at 72° C. for 2 min. The amplified PCR product was cleaved with Nhe I and Sma I restriction endonucleases and ligated into the same restriction sites (Nhe I and Sma I recognition sites) of the pRNAT/shMDR vector, using T4 ligase (Invitrogen, USA), thereby constructing a recombinant vector, designated pRNAT/shMDR(Sal).

Thereafter, PCR amplification of a TK-GFP fusion cDNA was carried out using the pTK-GFP vector as a template and primers of SEQ ID NOS: 7 and 11. PCR was carried out as follows: initial denaturation of template cDNA at 94° C. for 2 min, followed by 30 cycles of denaturation at 94° C. for 30 seconds, annealing at 60° C. for 30 seconds and extension at 72° C. for 2 min. The amplified PCR product was cleaved with Nhe I and Sal I restriction endonucleases and ligated into the same restriction sites (Nhe I and Sal I recognition sites) of the pRNAT/shMDR(Sal) vector, using T4 ligase (Invitrogen, USA), thereby cloning a recombinant vector containing MDR1 shRNA downstream of the U6 promoter and a TK-GFP fusion gene downstream of the CMV promoter. This vector was designated as shMDR-TK-GFP.

The recombinant vector of the present invention may be introduced into a host cell, using a conventional method known in the art. Preferably, intracellular incorporation of the vector into the host cell may be carried out by a conventional method known in the art, such as calcium chloride, microprojectile bombardment, electroporation, PEG-mediated fusion, microinjection, liposome-mediated method, and the like.

Examples of the host cell that can be utilized in the present invention may include, but are not limited to, prokaryotic cells such as Escherichia coli, Bacillus subtilis, Streptomyces, Pseudomonas, Proteus mirabilis, and Staphylococcus, lower eukaryotic cells such as fungi (e.g. Aspergillus), yeast (e.g. Pichia pastoris), Saccharomyces cerevisiae, Schizosaccharomyces, and Neurospora crassa, and higher eukaryotic cells such as insect cells, plant cells, mammalian cells. Preferably, the host cell may be human cells.

Meanwhile, standard recombinant DNA and molecular cloning techniques used in the present invention are well known in the art and can be found in the following literature: Sambrook, J., Fritsch, E. F. and Maniatis, T., Molecular Cloning: A Laboratory Manual, 2nd ed., Cold Spring Harbor Laboratory: Cold Spring Harbor, N.Y. (1989); Silhavy, T. J., Bennan, M. L. and Enquist, L. W., Experiments with Gene Fusions, Cold Spring Harbor Laboratory: Cold Spring Harbor, N.Y. (1984); and Ausubel, F. M. et al., Current Protocols in Molecular Biology, published by Greene Publishing Assoc. and Wiley-Interscience (1987).

A human colon cancer cell line transfected with the recombinant vector of the present invention exhibited superior drug sensitivity upon combined use of ganciclovir and an anticancer drug, as compared to separate use of each drug. Therefore, the present invention provides a pharmaceutical composition for treating neoplastic diseases, comprising the recombinant vector of the present invention, ganciclovir and an anticancer drug.

There is no particular limit to the anticancer drug which will be used in combination with the recombinant vector of the present invention, as long as it is a conventional anticancer drug known in the art, and it is movable by p-glycoprotein. Preferred examples of the anticancer drug may include paclitaxel, doxorubicin, vincristine, daunorubicin, vinblastine, actinomycin-D, docetaxel, bisantrene, homoharringtonine, Gleevec (STI-571), and the like. Preferably, the anticancer drug may be used in combination with ganciclovir. Specifically, ganciclovir may be used in combination with an anticancer drug selected from the group consisting of paclitaxel, doxorubicin and vincristine.

Examples of the neoplastic diseases that can be treated by the present invention may include diseases with pathological symptoms of tumors or malignancies, preferably such as colon cancer, hepatoma, leukemia, lymphoma, multiple myeloma, chronic myelogenous leukemia (CML), neuroblastoma, and the like. More preferably, the neoplastic disease may be colon cancer.

The pharmaceutical composition according to the present invention may comprise a therapeutically effective amount of the recombinant vector of the present invention and an anticancer drug alone or in combination with one or more pharmaceutically acceptable carriers. As used herein, the term “therapeutically effective amount” refers to an amount which is capable of producing the desired therapeutic response greater than that exhibited by a negative control. Preferably, the therapeutically effective amount is a dose sufficient to prevent or treat the neoplastic disease.

A therapeutically effective amount of the recombinant vector and anticancer drug in the present invention may be in a range of 0.0001 to 100 mg/day/kg (BW), preferably 0.01 to 1 mg/day/kg. However, an effective dose of the drug may vary depending upon various factors such as kinds and severity of disease, age, weight, health and sex of patients, administration routes and treatment duration.

As used herein, the term “pharmaceutically acceptable” means that the compound is physiologically acceptable, and does not cause allergic reactions (such as gastrointestinal disorders, and vertigo) or similar reactions with no inhibitory effects on the action of an active ingredient, when it is administered to humans or animals. Examples of the pharmaceutically acceptable carrier may include all kinds of solvents, dispersion media, oil-in-water or water-in-oil emulsions, aqueous compositions, liposomes, microbeads and microsomes.

Meanwhile, the pharmaceutical composition of the present invention may be appropriately formulated in conjunction with any suitable carrier by a conventional method known in the art, depending upon administration routes of the drug. There is no particular limit to the administration route of the pharmaceutical composition. Therefore, the drug composition in accordance with the present invention may be administered via oral or parenteral routes. Examples of the parenteral administration route may include transdermal, intranasal, intraperitoneal, intramuscular, subcutaneous and intravenous routes.

When the pharmaceutical composition of the present invention is administered via an oral route, the pharmaceutical composition in conjunction with any orally acceptable vehicle may be formulated into various dosage forms such as powders, granules, tablets, pills, dragees, capsules, solutions, gels, syrups, suspensions, and wafers, according to a conventional method known in the art. Examples of suitable vehicles may include various kinds of fillers, for example sugars such as lactose, dextrose, sucrose, sorbitol, mannitol, xylitol, erythritol and maltitol; starches such as corn starch, wheat starch, rice starch and potato starch; cellulose substances such as cellulose, methyl cellulose, sodium carboxymethyl cellulose and hydroxypropyl methyl cellulose; gelatin, polyvinylpyrrolidone (PVP) and the like. If desired, there may be added disintegrating agents such as cross-linked polyvinylpyrrolidone, agar, and alginic acid or sodium alginate. Further, the pharmaceutical composition may further comprise anticoagulants, lubricants, wetting agents, fragrances, emulsifiers and preservatives.

When the pharmaceutical composition of the present invention is administered via a parenteral route, the pharmaceutical composition in conjunction with any parenterally acceptable vehicle may be formulated into, for example, an injectable preparation, a transdermal preparation or a nasal inhalant, according to a conventional method known in the art. Upon formulation of the injectable preparation, sterilization must be performed in conjunction with protection of the pharmaceutical preparation from microbial contamination including pathogenic bacteria and fungi. Examples of the vehicle suitable for the injectable preparation may include, but are not limited to, solvents or dispersion media including water, ethanol, polyols (such as glycerol, propylene glycol, and liquid polyethylene glycol), mixtures thereof and/or vegetable oil. More preferably, examples of the suitable vehicle may include isotonic solutions such as Hank's solution, Ringer's solution, PBS (phosphate buffered saline) containing triethanolamine, sterile water for injection, 10% ethanol, 40% propylene glycol and 5% dextrose. In order to protect the injectable preparation against microbial contamination, the preparation may further comprise various antibacterial and antifungal agents, for example, parabens, chlorobutanol, phenol, sorbic acid, thimerosal, and the like. In many cases, it will be preferable to include isotonic agents, for example, sugar or sodium chloride.

In the case of the transdermal formulation, the inventive pharmaceutical composition may be formulated in the form of ointments, creams, lotions, gels, external solutions, pastes, liniments, or aerosols. The term “transdermal administration” means that a therapeutically effective amount of an active ingredient contained in a pharmaceutical composition transmits into the skin when the pharmaceutical composition is topically applied to the skin. These formulations are described in the literature that is a guidebook generally known in all pharmaceutical chemistry fields (Remington's Pharmaceutical Sciences, 15^{th} Edition, 1975, Mack Publishing Company, Easton, Pa.).

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gases. In the case of a pressurized aerosol, the dosage unit may be determined by providing a valve to deliver a metered amount. Capsules and cartridges of, e.g., gelatin for use in an inhaler or insufflator may be formulated containing a powdered mixture of the compound and a suitable powder base such as lactose or starch.

Other pharmaceutically acceptable vehicles can be found in the literature (Remington's Pharmaceutical Sciences, 19^{th} ed., Mack Publishing Company, Easton, Pa., 1995).

The pharmaceutical composition of the present invention may further comprise one or more buffers (e.g. saline or PBS), carbohydrates (e.g. glucose, mannose, sucrose or dextran), antioxidants, bacteriostatic agents, chelating agents (e.g. EDTA or glutathione), adjuvants (e.g. aluminum hydroxide), suspending agents, thickening agents, and/or preservatives.

Additionally, the pharmaceutical composition of the present invention may be appropriately formulated by a conventional method known in the art, such that it is possible to achieve fast, sustained or delayed release of active ingredients after administration of the composition to a mammal.

Further, the pharmaceutical composition of the present invention may be administered in combination with a known drug having therapeutic effects for treating neoplastic diseases.

Further, the present invention provides a method for imaging a neoplastic lesion, comprising:

(a) transfecting a tumor cell with the recombinant vector of the present invention; and

(b) detecting fluorescence of the transfected tumor cell.

Since the recombinant vector of the present invention is constructed such that a host cell expresses GFP (green fluorescent protein), tumor cells transfected with the recombinant vector of the present invention can emit fluorescence. There is no particular limit to the fluorescence detection method. Therefore, detection of fluorescence can be made by any conventional method known in the art.

In one embodiment of the present invention, the inventors of the present invention constructed a novel recombinant vector, designated shMDR-TK-GFP, which is effective for gene therapy of tumor by the action of two different target genes contained in the expression vector and which is also capable of achieving tumor lesion imaging. Such effects were achieved by combination of a Herpes simplex virus-thymidine kinase (HSV-tk) gene, a GFP (green fluorescent protein) gene and MDR1 shRNA into one vector system and consequent intracellular incorporation of the thus-constructed vector into a host cell. Further, the present inventors established a human colon cancer cell line transfected with the aforesaid vector (see Example 1).

In another embodiment of the present invention, whether MDR1 shRNA is effectively expressed in the aforesaid transfected colon cancer cell line was investigated. As a result, it was confirmed that the transfected colon cancer cell line exhibits a decreased level of the MDR1 mRNA and a decreed expression level of P-glycoprotein which is the expression product of the MDR1 gene, as compared to a control cell line (see Example 2).

In another embodiment of the present invention, whether a thymidine kinase-GFP (TK-GFP) fusion protein is effectively expressed in the aforesaid transfected colon cancer cell line was investigated. As a result, it was confirmed that the transfected colon cancer cell line exhibits expression of the aforesaid fusion protein which was not present in the control cell line (see Example 3). Taken together, it was confirmed that the inventive vector (shMDR-TK-GFP recombinant vector) is a vector capable of providing effective co-expression of the MDR1 shRNA and the TK-GFP fusion protein.

Further, in order to confirm whether the MDR1 shRNA and thymidine kinase expressed in the transfected colon cancer cell line of the present invention are effectively functional, accumulation of the anticancer drug and drug sensitivity to an anticancer drug were examined for the transfected cell line MTKG which will be illustrated in Example 1, according to another embodiment of the present invention. As a result, it was confirmed that the MTKG cell line exhibits significantly higher intracellular accumulation of an isotope-labeled anticancer drug simultaneously with increased drug sensitivity to the anticancer drug, as compared to the control cell line (see Example 4). From these results, intracellular incorporation of MDR1 shRNA leads to inhibition of P-glycoprotein expression to thereby effectively increase the intracellular accumulation of the anticancer drug and the anticancer drug susceptibility.

In another embodiment of the present invention, ganciclovir accumulation and drug sensitivity were investigated in the MTKG cell line. As a result, it was confirmed that the MTKG cell line exhibits significantly higher intracellular accumulation of ganciclovir simultaneously with increased drug sensitivity to ganciclovir, as compared to the control cell line (see Example 5).

In another embodiment of the present invention, effects of co-administration of ganciclovir with the anticancer drug on cancer cells were examined in the transfected cell line. For this purpose, the MTKG cells were administered with ganciclovir and anticancer drug for a given period of time, and effects of these drugs on cells were investigated. As a result, it was confirmed that co-administration of ganciclovir and anticancer drug exhibits pronounced effects, as compared to separate administration of each drug (see Example 6).

As discussed above, the present inventors have succeeded in cloning of a vector with co-expression of MDR1 shRNA, thymidine kinase and GFP protein for the first time in the world. Further, it was demonstrated that co-administration of ganciclovir with the anticancer drug through the intracellular incorporation of such an expression vector exhibits pronounced anticancer effects on cancer cells, as compared to independent administration of each drug.

In conclusion, the present invention relates to a recombinant vector capable of expressing MDR1 shRNA and thymidine kinase, and a use thereof.

### EXAMPLES

Now, the present invention will be described in more detail with reference to the following Examples. These examples are provided only for illustrating the present invention and should not be construed as limiting the scope and spirit of the present invention.

### Example 1

### Cloning of Vector with Co-Expression of MDR1 shRNA and Thymidine Kinase Gene

1-1. Construction of HSV-Thymidine Kinase/GFP Fusion Gene

For construction of a fusion gene of a Herpes simplex virus-thymidine kinase (HSV-tk) gene and a GFP (green fluorescent protein) gene (hereinafter, referred to as “TK-GFP gene”), PCR amplification was carried out using HSV-tk cDNA (by courtesy of Dr. Jae Yong Park, Department of Internal Medicine, Division of Pulmonary, Medicine College of Kyungpook National University, Korea) as a template, and primers having sequences as set forth in SEQ ID NOS: 7 and 8. PCR was carried out as follows: initial denaturation of template cDNA at 94° C. for 2 min, followed by 30 cycles of denaturation at 94° C. for 30 seconds, annealing at 60° C. for 30 seconds and extension at 72° C. for 1 min. The amplified PCR product was cleaved with Nhe I and BamH I restriction endonucleases and ligated into the same restriction sites (Nhe I and BamH I recognition sites) of a pEGFP-C1 vector (Clontech, USA), using T4 ligase (Invitrogen, USA), thereby constructing a recombinant vector, designated pTK-GFP.

1-2. Construction of Triple Expression Vector

First, in order to construct a vector where MDR1 shRNA is expressed, a nucleotide sequence (5′-GGCCUAAUGCCGAACACAU-3′) of SEQ ID NO: 6 was used as a target sequence. In order to construct an insert fragment which allows expression of shRNA for the aforesaid target sequence in an expression vector of the present invention, a pair of primers (SEQ ID NOS: 2 and 3) were designed. The primer sequences of SEQ ID NOS: 2 and 3 were designed to contain both sense and antisense nucleotide sequences for a nucleotide sequence of SEQ ID NO: 6, and insert a nucleotide sequence consisting of 4 bases (CGAA) therebetween to form a loop.

The thus-designed primer set was synthesized by Bionics (Seoul, Korea). Then, each 200 pmol of the primer fragments as set forth in SEQ ID NOS: 2 and 3 was dissolved in STE buffer (10 mM Tris pH 8.0, 50 mM NaCl, 1 mM EDTA). The resulting solution was heated at 95° C., followed by slow cooling to allow binding of two primers, thereby constructing an insert fragment. The thus-formed insert fragment was inserted into BamH I and Xho I restriction sites of pRNAT/U6 vector (GenScript, USA), using T4 ligase (Invitrogen, USA), thereby constructing a recombinant vector, designated pRNAT/shMDR.

In order to make a Sal I restriction site for insertion of the TK-GFP fusion gene simultaneously with removal of a GFP (Green fluorescent protein) gene present in the pRNAT/shMDR vector, PCR amplification was carried out using a pair of primers (SEQ ID NOS: 9 and 10) and the pRNAT/shMDR vector as a template. PCR was carried out as follows: initial denaturation of template DNA at 94° C. for 2 min, followed by 30 cycles of denaturation at 94° C. for 30 seconds, annealing at 60° C. for 30 seconds and extension at 72° C. for 2 min. The amplified PCR product was cleaved with Nhe I and Sma I restriction endonucleases and ligated into the same restriction sites (Nhe I and Sma I recognition sites) of the pRNAT/shMDR vector, using T4 ligase (Invitrogen, USA), thereby constructing a recombinant vector, designated pRNAT/shMDR(Sal).

Thereafter, PCR amplification of a TK-GFP fusion cDNA was carried out using the pTK-GFP vector as a template and primers of SEQ ID NOS: 7 and 11. PCR was carried out as follows: initial denaturation of template cDNA at 94° C. for 2 min, followed by 30 cycles of denaturation at 94° C. for 30 seconds, annealing at 60° C. for 30 seconds and extension at 72° C. for 2 min. The amplified PCR product was cleaved with Nhe I and Sal I restriction endonucleases and ligated into the same restriction sites (Nhe I and Sal I recognition sites) of the pRNAT/shMDR(Sal) vector, using T4 ligase (Invitrogen, USA), thereby cloning a recombinant vector containing MDR1 shRNA downstream of the U6 promoter and a TK-GFP fusion gene downstream of the CMV promoter. This vector was designated as shMDR-TK-GFP. The constructed vector “shMDR-TK-GFP” was sequenced using a DNA sequencer (Model No. AB13700, manufactured by Applied Biosystems, USA) to thereby confirm whether the correct cloning was made as desired (results not shown).

TABLE 1
Primers used for cloning of shMDR-TK-GFP

| | | |
|---|---|---|
| | | SEQ ID |
| Primers | Sequences | NO |

| | | |
|---|---|---|
| HSV-tk sense | 5′-aaa agc tag cct tgg tgg cgt gaa ac-3′ | 7 |
| HSV-tk antisense | 5′-aaa agg atc cga gtt agc ctc ccc ca-3′ | 8 |
| shMDR1 sense | 5′-gat ccc ggc cta atg ccg aac aca tcg | 2 |
| | aaa tgt gtt cgg cat tag gcc ttt ttt cca | |
| | ac-3′ | |
| shMDR1 antisense | 5′-tcg agt tgg aaa aaa ggc cta atg ccg | 3 |
| | aac aca ttt cga tgt gtt cgg cat tag gcc | |
| | gg-3′ | |
| Nhe-Sal sense | 5′-aaa agc tac cgt cga cta gat aac tga | 9 |
| | act tg-3′ | |
| Nhe-Sal antisense | 5′-tct tga tca gat ccg aaa atg g-3′ | 10 |
| TK-GFP antisense | 5′-aaa agt cga ctt act tgt aca gct cgt | 11 |
| | cca t-3′ | |

1-3. Transfection of HCT-15 Cells

The human colon cancer cell line HCT-15 (obtained from Korean Cell Line Bank (KCLB), Seoul, Korea) was transfected with the expression vector shMDR-TK-GFP constructed in Section 1-2.

The HCT-15 cells were cultured in RPMI 1640 medium supplemented with 20% heat-inactivated fetal bovine serum (FBS), 100 units/mL of penicillin G and 100 μg/mL of streptomycin. Transfection of the HCT-15 cells with the recombinant expression vector shMDR-TK-GFP was carried out using the Lipofectamine reagent (Invitrogen, USA) according to the manufactures instructions. 48 hours after transfection, the cells were treated with 500 μg/mL of geneticin (G418, Invitrogen, USA) and cultured for 10 to 12 days to pick drug-resistant colonies. The thus-transfected cells were selected and designated as MTKG. As a negative control (HCT/Mock), cells transfected with pRNAT/U6 which is a vector containing no shMDR-TK-GFP gene were used.

### Example 2

### Expression of MDR1 shRNA in Transfected Cells

2-1. Analysis of mRNA Expression

Expression of MDR1 shRNA in MTKG cells of Section 1-3 of Example 1 was examined by RT-PCR For this purpose, total RNA was isolated from HCT-15, HCT/Mock, and MTKG cells, using Trizol reagent (Invitrogen) according to the manufacturer's instructions. Then, cDNA was prepared using 2 μg of the isolated total RNA as a template, and Oligo(dT)₁₅ primer (Bionics, Seoul, Korea) and reverse transcriptase (Promega). PCR amplification for MDR1 mRNA was carried out using the prepared cDNA as a template and a pair of primers (SEQ ID NOS: 12 and 13). PCR was carried out as follows: initial denaturation of template cDNA at 94° C. for 2 min, followed by 30 cycles of denaturation at 94° C. for 30 seconds, annealing at 60° C. for 30 seconds and extension at 72° C. for 1 min.

TABLE 2
Primers for MDR1 amplification

| | | |
|---|---|---|
| | | SEQ ID |
| Primers | Sequences | NO |

| | | |
|---|---|---|
| MDR1 sense | 5′-gga gtg tcc gtg gat cac | 12 |
| | a-3′ | |
| MDR1 antisense | 5′-aat aca tca ttg cct ggg | 13 |
| | tga ag-3′ | |

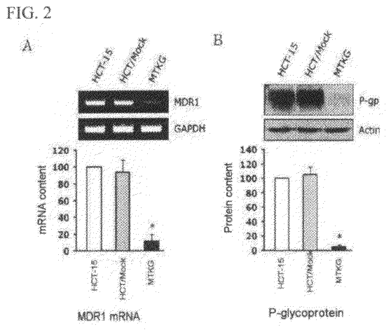

From the experimental results shown in FIG. 2A, it was confirmed that the MTKG cells exhibit a decreased level of MDR1 mRNA. Therefore, this fact represents that expression of MDR1 shRNA resulted in a decreased level of MDR1 mRNA.

2-2. Analysis of Protein Expression

In order to investigate whether intracellular incorporation of MDR1 shRNA inhibits expression of P-glycoprotein which is an expression product of the MDR1 gene, Western blot analysis was carried out for proteins isolated from HCT-15, HCT/Mock, and MTKG cells, respectively. The procedure will be briefly described as follows. HCT-15, HCT/Mock, and MTKG cells were lysed in a cell lysis buffer (containing 50 mM Tris-HCl, 150 mM NaCl, 1% Triton X-100, 1 mM CaCl₂, 1 mM MgC₂ and protease inhibitor cocktail (Roche, USA), pH 7.4). 30 μg of each protein in the cell lysates was subjected to electrophoresis on polyacrylamide gel containing 7% SDS, and the developed protein gel was transferred to a nitrocellulose membrane. Thereafter, anti-P-glycoprotein antibodies (clone C219, Calbiochem, USA) were diluted in a TBS-T solution (50 mM Tris-HCl, pH 7.6, 150 mM NaCl, 0.1% Tween 20) and were allowed to react and bind with a nitrocellulose membrane for more than 16 hours under refrigeration conditions. After the reaction was complete, the membrane was washed three times with a TBS-T solution, followed by binding reaction with horseradish peroxidase (HRP)-conjugated anti-mouse IgG antibodies (Santa Cruz, USA) for 1 hour at room temperature. After the reaction was complete, the membrane was washed the times with a TBS-T solution, and the region where an antigen-antibody reaction took place was visualized with addition of 1 mL of a chemiluminescence reagent (ECL™, Amersham, USA), followed by exposure to X-ray film. As a control group, the nitrocellulose membrane was reacted with anti-actin antibodies (Sigma, USA).

From the experimental results shown in FIG. 2B, it was confirmed that the MTKG cells exhibit a decreased expression level of P-glycoprotein, similar to the results of Section 2-1.

### Example 3

### Expression of TK-GFP Gene in Transfected Cells

3-1. Analysis of Protein Expression

In order to examine expression of an intracellularly incorporated TK-GFP gene, Western blot analysis was carried out in the same manner as in Section 2-2 of Example 2, except that anti-GFP antibodies (clone B-2, Santa Cruz, USA) were used for proteins isolated from HCT-15, HCT/Mock, and MTKG cells.

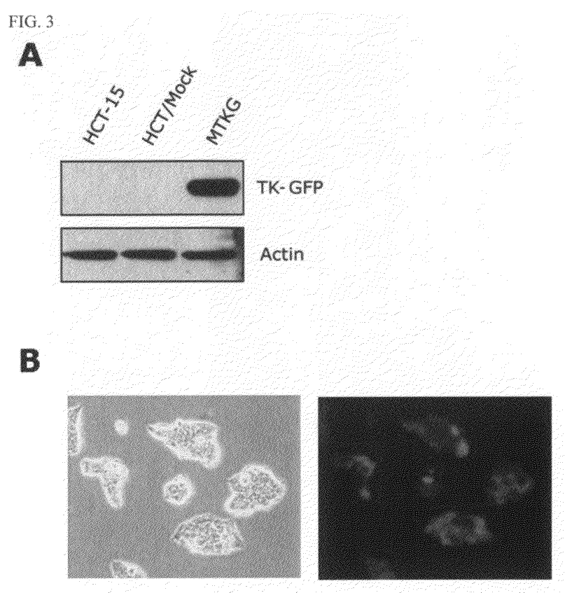

From the experimental results shown in FIG. 3A, it can be seen that the MTKG cells exhibit significant expression of the TK-GFP fusion protein, as compared to a control group.

3-2. Fluorescence Analysis

In order to further examine expression of an intracellularly incorporated TK-GFP gene, MTKG cells were observed under a light microscope and a fluorescence microscope, respectively.

From the experimental results shown in FIG. 3B, it can be seen that all the cells observed under the light microscope exhibited the fluorescence of the GFP protein upon examination of the cells under the fluorescence microscope.

### Example 4

### Functional Identification by MDR1 shRNA

4-1. Intracellular Accumulation of Anticancer Drugs

A degree of intracellular drug accumulation by MDR1 shRNA was investigated. MTKG cells constructed in Section 1-3 of Example 1, and HCT-15 and HCT/Mock cells as a control group were cultured in 6-well plates containing RPMI 1640 medium for 48 hours, to which 50 nM of an isotope-labeled anticancer drug ([³H]-paclitaxel, Moravek, USA) was then added, followed by incubation at 37° C. for 2 hours. Then, the plate was washed three times with a phosphate buffer, and a cell lysis buffer (50 mM Tris-HCl, 150 mM NaCl, and 1% SDS) was added thereto to lyse the cells. For each cell lysate sample, an isotope activity was measured using a beta-counter.

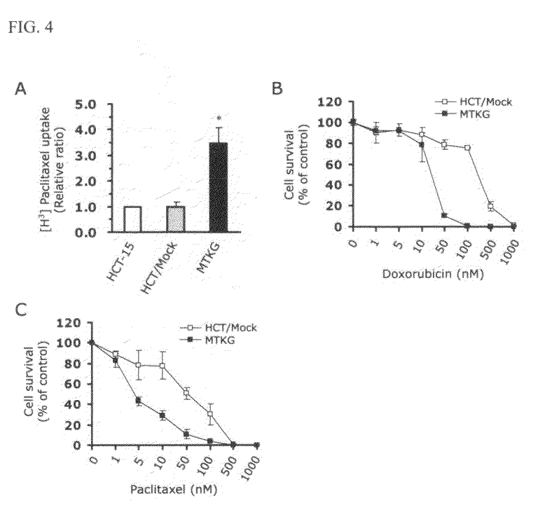

From the experimental results shown in FIG. 4A, it was confirmed that MTKG cells expressing MDR shRNA exhibit higher accumulation of paclitaxel, as compared to HCT-15 and HCT/Mock cells as a control group.

4-2. Test for Sensitivity to Anticancer Drugs

Whether the anticancer drug sensitivity of MTKG cells is enhanced due to increased intracellular accumulation capacity of the anticancer drug was confirmed by clonogenic assay.

First, MTKG cells and HCT/Mock cells as a control group were inoculated at a cell density of 10³ cells/well in a 6-well plate containing RPMI 1640 medium, followed by addition of an anticancer drug (doxorubicin or paclitaxel) at different concentrations (0, 1, 5, 10, 50, 100, 500, and 1000 nM) and incubation for 10 days. Thereafter, the culture medium was eliminated, and the cells were washed three times with a phosphate buffer and stained with a dye (Diff Quick staining kit). Colony formation was confirmed and colony count was made.

As shown in FIG. 4B, it was confirmed that the HCT/Mock cells as a control group exhibit 90% or higher inhibition of cell proliferation at a doxorubicin concentration of 500 nM or higher, whereas MTKG cells exhibit 90% or higher inhibition of cell proliferation at a doxorubicin concentration of 50 nM. Further, as shown in FIG. 4C, it was confirmed that the HCT/Mock cells exhibit 70% or higher inhibition of cell proliferation at a paclitaxel concentration of 100 nM, whereas MTKG cells exhibit 70% or higher inhibition of cell proliferation at a paclitaxel concentration of 10 nM. Therefore, the above experimental results represent nearly a ten-fold increase in the anticancer drug sensitivity.

### Example 5

### Functional Identification by TK-GFP Protein

5-1. Intracellular Accumulation of Ganciclovir

A degree of intracellular ganciclovir accumulation by the TK-GFP gene was investigated as follows. MTKG cells constructed in Section 1-3 of Example 1, and HCT-15 and HCT/Mock cells as a control group were cultured in 6-well plates containing RPMI 1640 medium for 48 hours, to which 0.76 μCi/mL of an isotope-labeled ganciclovir ([H³]-Ganciclovir, Moravek, USA) was then added, followed by incubation at 37° C. for 45 min. Then, the plate was washed three times with a phosphate buffer and a cell lysis buffer (50 mM Tris-HCl, 150 mM NaCl, and 1% SDS) was added thereto to lyse the cells. For each cell lysate sample, an isotope activity was measured using a beta-counter.

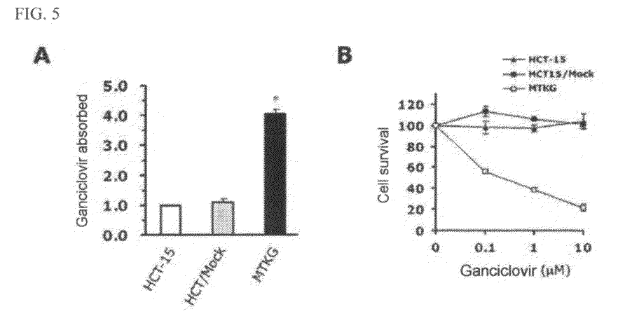

From the experimental results shown in FIG. 5A, it was confirmed that MTKG cells with expression of the TK-GFP gene exhibit higher accumulation of ganciclovir, as compared to HCT-15 and HCT/Mock cells as a control group.

5-2. Test for Drug Sensitivity to Ganciclovir

Whether increased intracellular accumulation capacity of ganciclovir confirmed in Section 5-1 leads to the enhanced drug sensitivity of MTKG cells to ganciclovir was examined by a cell proliferation assay using CellTiter 96 Aqueous One Solution cell proliferation assay kit (Promega, USA), First, MTKG cells and HCT/Mock cells as a control group were inoculated at a cell density of 10² cells/well in a 96-well plate containing RPMI 1640 medium, followed by addition of ganciclovir at different concentrations (0, 0.1, 1, and 10 μM) and incubation for 4 days. Thereafter, 20 μl of detection solution were added to each well. The cells were incubated for 90 min at 37° C. and then measured absorbance at 490 nm using miroplate reader (BioRad, USA). From the experimental results shown in FIG. 5B, it was confirmed that proliferation of HCT-15 and HCT/Mock cells as a control group was not affected by administration of ganciclovir, whereas proliferation of the MTKG cells was affected even at a ganciclovir concentration of 0.1 μM or higher, thus representing increased drug sensitivity of the cell.

### Example 6

### Effects of Co-Administration of Ganciclovir and Doxorubicin on Cells

In order to confirm effects of co-administration of an anti-viral drug ganciclovir and an anticancer drug (doxorubicin) on cancer cells, clonogenic assay was carried out. For this purpose, first, MTKG cells were inoculated and cultured in a 100 mm plate containing RPMI 1640 medium, and the cells were then washed three times with a fresh medium. The cells were desorbed from the plate using trypsin-EDTA, and 1×10³ cells were counted and inoculated into a 6-well plate with addition of ganciclovir (0.1 μCi) and doxorubicin at two different concentrations (10 and 25 nM), followed by incubation. 10 days after incubation, the culture medium was eliminated, and the cells were washed three times with a phosphate buffer and stained with a dye (Diff Quick staining kit). Colony formation was confirmed and colony count was made.

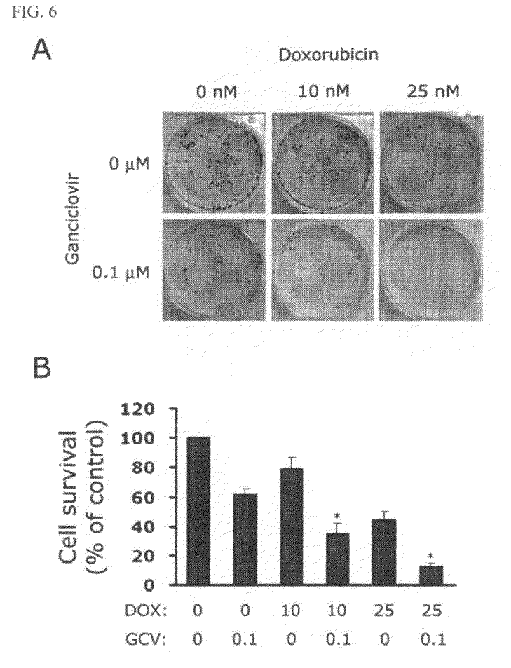

From the experimental results shown in FIG. 6, it was confirmed that the MTKG cells with intracellular incorporation of the shMDR-TK-GFP vector exhibit a lower cell survival rate upon combined treatment of doxorubicin and ganciclovir on cells, as compared to separate administration of each drug, thus representing that co-administration of doxorubicin and ganciclovir leads to significantly greater killing of tumor cells. That is, co-administration of 10 nM doxorubicin and 0.1 μCi ganciclovir resulted in a significantly lower cell survival rate of 34.9±10.0%, as compared to that of 61.1±4.1% achieved upon administration of 0.1 μCi ganciclovir alone and that of 78.8±7.8% achieved upon administration of 10 nM doxorubicin alone. Further, co-administration of 25 nM doxorubicin and 0.1 μCi ganciclovir also resulted in a significantly lower cell survival rate of 11.2±1.2%, as compared to that of 61.1±4.1% achieved upon administration of 0.1 μCi ganciclovir alone and that of 37.7±3.3% achieved upon administration of 25 nM doxorubicin alone.

Taken together, it can be seen that the intracellular incorporation of a shMDR-TK-GFP vector leads to more effective tumor cell death upon co-administration of the anti-viral drug ganciclovir with the anticancer drug doxorubicin than separate administration of each drug.

As apparent from the above description, the recombinant vector of the present invention is capable of achieving efficient expression of MDR1 shRNA and a thymidine kinase-GFP fusion protein within the host cell and is therefore highly effective for combined therapy of anticancer drugs. Further, the recombinant vector of the present invention enables imaging of neoplastic lesions. Therefore, the recombinant vector of the present invention can be used in combination with other anticancer drugs for treatment of neoplastic diseases.

1. A recombinant vector comprising a polynucleotide encoding a first promoter and MDR1 shRNA being operatively linked to the first promoter; and a second promoter and HSV thymidine kinase being operatively linked to the second promoter.

2. The vector according to claim 1, wherein the MDR1 shRNA comprises a nucleotide sequence as set forth in SEQ ID NO: 2.

3. The vector according to claim 1, wherein the MDR1 shRNA comprises a nucleotide sequence as set forth in SEQ ID NO: 3.

4. The vector according to claim 1, wherein the HSV thymidine kinase comprises an amino acid sequence as set forth in SEQ ID NO: 4.

5. The vector according to claim 1, wherein the polynucleotide comprises a nucleotide sequence as set forth in SEQ ID NO: 5.

6. The vector according to claim 1, wherein the vector is a shMDR-TK-GFP vector having a cleavage map comprising a polynucleotide encoding a U6 promoter and a MDR1 shRNA being operatively linked to the U6 promoter: a CMV promoter and an HSV thymine kinase being operatively linked to the CMV promoter; and an SV40 promoter and a neomycin resistance gene being operatively linked to the SV40 promoter.

7. A cell line transfected with the vector of claim 1.

8. The cell according to claim 6, wherein the cell is a human cell.

9. A pharmaceutical composition for treating neoplastic diseases, comprising the recombinant vector of claim 1, ganciclovir, and an anticancer drug.

10. The composition according to claim 8, wherein the anticancer drug is selected from the group consisting of paclitaxel, doxorubicin, and vincristine.

11. A method for imaging a neoplastic lesion, comprising:
(a) transfecting a tumor cell with the recombinant vector of claim 1; and
(b) detecting fluorescence of the transfected tumor cell.
